(19) United States
(12) Patent Application Publication
(10) Pub. No.: US 2024/0359383 A1
(43) Pub. Date: Oct. 31, 2024

(54) METHOD FOR APPLYING ELASTOMER AND A CABLE TO A FABRIC LAYER

(71) Applicant: NTT NEW TEXTILE TECHNOLOGIES GMBH, BALINGEN (DE)

(72) Inventor: HANS BAUER, BALINGEN (DE)

(73) Assignee: NTT NEW TEXTILE TECHNOLOGIES GMBH, BALINGEN (DE)

(21) Appl. No.: 18/762,928

(22) Filed: Jul. 3, 2024

Related U.S. Application Data
(63) Continuation-in-part of application No. PCT/EP2023/025140, filed on Mar. 29, 2023.

(30) Foreign Application Priority Data
Apr. 14, 2022 (DE) 10 2022 109 327.1

(51) Int. Cl. B29C 48/00; A61F 13/00; A61F 13/01; B29C 48/05; B29C 48/154; B29C 48/34; B29K 619/00; B29K 705/00; B29L 31/00; B29L 31/48

(52) U.S. Cl. CPC B29C 48/0021; A61F 13/00995; A61F 13/01008; A61F 13/01038; B29C 48/05; B29C 48/154; B29C 48/34; A61F 2013/00119; B29K 2619/00; B29K 2705/00; B29K 2713/00; B29K 2995/0005; B29K 2995/0046; B29L 2031/48; B29L 2031/753

(57) ABSTRACT

In a method for supplying elastomer and a cable embedded in the elastomer to a fabric layer of a garment or a bandage, the elastomer is applied in a flowable state to the fabric layer via a nozzle and the cable is applied to the fabric layer via the same nozzle simultaneously with the elastomer, at least in some phases such that the elastomer encases at least part of the cable.

### CROSS-REFERENCE TO RELATED APPLICATIONS

This is a continuation-in-part application of pending international patent application PCT/EP2023/025140 filed Mar. 29, 2023 and claiming the priority of German patent application No. 10 2022 109 327.1 filed Apr. 14, 2022. Both the said international patent application PCT/EP2023/025140 and the said German patent application No. 10 2022 109 327.1 are incorporated herein by reference in their entireties as though fully set forth.

### BACKGROUND OF THE INVENTION

The invention resides in a method for applying elastomer and a cable embedded into the elastomer to a fabric layer of a garment or to a bandage.

EP 3 172 977 A1 discloses a method for producing articles of clothing or bandages wherein a fabric layer of the clothing or, respectively, the bandage is provided with a combination of elastomer and reinforcement material. Herein, the reinforcement material is applied in a liquid or soft deformable state and cures after application. The elastomer layer and the reinforcement material are applied each via a nozzle wherein the nozzle for the reinforcement material is in form of a 3-D pressure head.

US 2017/0151770 A1 discloses the application of an elastomer for a fabric layer with the aid of a pressure head.

DE 10 2018 104 774 B3 discloses an electrode which may be wearable in contact with the skin when integrated into a garment or a bandage. The electrode comprises an electrically conductive skin contact layer which can provide a connection to an electric power supply via an electrically conductive connector.

DE 10 2020 200 508 A1 discloses a pressure head for a pressure system for the additive production of a fiber construction component. The pressure head comprises a first supply section for supplying cord-shaped thermoplastic material into the pressure head and a second supply section for supplying cord-shaped fiber material or conductor material forming core material to the pressure head. Furthermore, a heating arrangement for plasticizing the thermoplast material supplied through the first supply section and a cutting arrangement for cutting the core material supplied through the second supply section are provided. Via a nozzle of the pressure head, which forms an extrusion channel, the core material is embedded into the thermoplastic material.

US 2015/0165666 A1 discloses an arrangement including a nozzle for the co-extrusion of a polymer fiber and a fiber strand.

EP 3 130 444 A1 discloses a 3D printer wherein a reinforcement filament with a core and a matrix material surrounding the core is introduced into an extrusion nozzle.

EP 0 187 270 A1 discloses a method for fixing an elastic ribbon or an elastic foil to an areal material. By co-extrusion or successive extrusion, a product is produced which consists of a hot melt layer and layer of a thermoplastic elastomer material.

DE 197 44 527 A1 discloses the production of an insulated electric conductor by an extrusion process. The extrusion is performed via an outer nozzle in which an inner nozzle is arranged.

It is the object of the present invention to apply, in a simple and efficient manner, an elastomer and a cable embedded into the elastomer onto a fabric layer of a garment or a bandage.

### SUMMARY OF THE INVENTION

The object is solved by the method including the features of the independent claim 1. The dependent claims are directed to expedient embodiments.

With the method according to the invention elastomer and a cable can be deposited on a fabric layer of a garment or a bandage in a single method step in such a way that the cable is partially or completely surrounded by the elastomer. The method is performed by means of a nozzle which is designed to apply the elastomer and to apply also the cable to the fabric layer. Herein the elastomer is applied in a deformable state via the nozzle and, at the same time, also the cable is applied via this nozzle to the fabric layer. As a result, only a single nozzle is needed for the application of the elastomer as well as the cable. The cable is deposited on the fabric layer into an elastomer path which is generated by the elastomer discharged from the nozzle. The elastomer and the cable are discharged from the nozzle at the same time.

After completion of the method step, basically, no additional treatment or additional process step is to be performed. The cable is at least partially surrounded whereby, on one hand, a secure and strong connection between the cable and the fabric layer is obtained and, on the other hand, the cable is protected from other influences such as humidity. The connection between the elastomer and the fabric layer occurs because the elastomer is applied to the fabric layer in a deformable or, respectively soft, flowable state so that the elastomer can penetrate into the fabric layer. After hardening, the elastomer is firmly joined with the fabric layer.

The garment of which the fabric material is part is for example an underwear clothing piece or an outer piece of clothing such as a shirt, a T-shirt, a pants such as a sport pants, a bra, or similar. Furthermore, it may be a girdle, a breast belt or a sleeve which is worn for example to cover the hand joint or the foot joint. In connection with the invention also these items are considered to be clothing pieces. The material layer may also be part of a bandage, in particular a joint bandage like for example a knee bandage or an elbow bandage.

Advantageously, the cable is electrically conductive so that accordingly, it can transmit electrical signals. This makes it possible to integrate for example electrodes or sensors into the garment or, respectively, the bandage so that electrical signals can be transmitted from the sensor or respectively the electrode via the cable that is connected to the sensor or respectively the electrode to an evaluation unit for an examination of the signals. With the sensors or, respectively, the electrode for example vital parameters of the body are determined based on voltage changes of the skin or a change of the electrical resistance of the skin. Vice versa it is also possible to use the sensor or, respectively, the electrode as a signal supplier via which electrical impulses which are generated in an impulse generator are transmitted to the skin for example for a muscle stimulation.

In a further advantageous embodiment, the elastomer and the cable exit via the same nozzle opening. It is therefore basically sufficient that the nozzle has only one discharge opening via which the elastomer as well as the cable exit. This embodiment has the advantage that the cable is at least partially surrounded by the elastomer when exiting the nozzle and, with the relative movement between the nozzle and the fabric layer, an elastomer strip with an integrated cable is produced.

Alternatively, it is also possible to provide for the cable and the elastomer a common nozzle which however has two discharge openings of which one is assigned to the cable and the other to the elastomer. In this case, the cable and the elastomer exit via different nozzle openings but which may be so arranged that the cable is embedded in the elastomer on the fabric layer in the desired way. Advantageously the exit opening for the elastomer is in the movement direction of nozzle arranged ahead of the exit opening for the cable so that in the movement direction first the elastomer and subsequently the cable are deposited.

With this method elastomer and the cable can be released from the nozzle at the same time. The method is performed in phases. The cable is deposited on the fabric layer via the nozzle in a further phase without elastomer. That is the discharge of elastomer is stopped for example by interrupting the elastomer supply to the nozzle. Bringing the cable out of the nozzle without any elastomer has the advantage that a section of the cable is disposed on the fabric layer free of any elastomer and at a slight distance from the fabric layer so that this section can easily be connected to an electrical component, in particular to the sensor or, respectively, the electrode or an evaluation unit. In particular, it is possible in this way to leave two opposite ends of the cable free of elastomer so that one end can be connected to the sensor or, respectively, the electrode and the opposite end can be connected to the evaluation unit. As the phase in which the cable but no elastomer is deposited on the fabric layer is at the beginning or at the end of an operating step, it is sufficient to provide only a single operating step with a relative movement between the cable and the fabric layer for bringing out the cable in phases with the elastomer wherein the cable is surrounded by elastomer and in phases wherein only the cable is brought out but no elastomer so that one or several sections of the cable remain without any elastomer.

Accordingly the method according to the invention wherein elastomer and a cable embedded into the elastomer is deposited on a fabric layer of a garment or a bandage, includes the following method steps:
The elastomer is deposited on the fabric layer via a nozzle in a deformable state,
The cable is deposited on the fabric layer via the same nozzle in phases together with the elastomer in such a way that the elastomer envelops the cable at least partially,
in another phase only the cable but no elastomer is deposited on the fabric layer.

Consequently the invention resides in a method for depositing elastomer and a cable embedded into the elastomer on a fabric material of a garment or a bandage, wherein the elastomer is deposited via a nozzle in a deformable state on the fabric layer and the cable is deposited via the same nozzle at the same time together with elastomer, but in phases in such a way that the elastomer at least partially surrounds the cable. In another additional phase, only the cable, but no elastomer, is deposited via the nozzle on the fabric layer.

In accordance with a further advantageous embodiment, an end of the cable which has already exited the nozzle is fixed relative to the fabric layer. It may be fixed either directly to the fabric layer or to a locally fixed item such as a table on which the fabric layer is disposed. The end of the cable exiting the nozzle is fixed before the method step in which in phases at the same time elastomer and the cable are deposited. As the cable is fixed, the cable can be pulled out of the nozzle opening by a relative movement between the nozzle and the fabric layer, that is when for example the nozzle is moved over the fabric layer. This procedure has the advantage that no drive arrangement for the movement of the cable is required.

In an alternative embodiment the cable may be moved by a drive arrangement at a speed which corresponds to the relative speed between the nozzle and the fabric layer. The drive arrangement may be disposed either in front of the nozzle so that the cable is guided into the nozzle or at the exit side of the nozzle so that the cable is pulled by the drive arrangement out of the nozzle. The drive arrangement has the advantage that the cable end already exited from the nozzle does not need to be fixed.

In accordance with a further advantageous embodiment the cable used is elastic in the longitudinal direction with a relatively high elasticity in particular to such an extent that it is expandable by a value of 1.6. Only an expansion of more than 1.6 times the expansion becomes non-linear. Up to this value a linear-elastic behavior of the cable expansion in its longitudinal direction is ensured. The elasticity of the cable permits the cable to follow expansions of the fabric material whereby the comfort of the wearer of the garment or the bandage provided with the cable is not detrimentally affected. Expansions of the fabric can also be followed by the elastomer by which the cable is at least partially surrounded and attached to the fabric layer.

The cable is surrounded by the elastomer preferably in such a way that, at the side facing away from the fabric, it is completely covered by the elastomer. The cable may be disposed directly on the fabric layer. Alternatively, the cable may be fully surrounded by elastomer that is elastomer is also provided on the side facing the fabric layer.

In accordance with another advantageous embodiment an elastic cable is used which consists of a rubber core surrounded by a metallic net. The rubber core supports the metallic net and permits an elastic extension in the longitudinal direction. The metal net, which surrounds the rubber core, is electrically conductive and facilitates the transmission of an electric signal while, at the same time, it improves the stability of the cable. Upon an expansion in the longitudinal direction also the metal net expands together with the rubber core. As soon as the rubber core contracts elastically the metal net contracts together with the rubber core.

As elastomer for example a thermoplastic elastomer is used which, when heated becomes soft or liquid and which hardens upon cooling. In a heated state the elastomer can be applied via a nozzle and, upon cooling, hardens and forms a firm connection with the fabric layer while protecting the cable. When hardened the elastomer has still same elasticity. As elastomer for example a silicon may be used.

Optionally, a flocking material may be applied to the elastomer after its application to the fabric layer and before its hardening, which improves the feeling of the wearer. The flocking material is adapted to absorb humidity.

The invention further relates to a garment or a bandage with a fabric layer provided with a cable which is connected to the fabric layer using an elastomer. The cable is of a design as described earlier, preferably electrically conductive and elastic in a longitudinal direction. The garment or, respectively, the bandage is manufactured preferably in accordance with the process described before.

At least one end of the cable, but preferably both ends of the cable, are free of elastomer while the cable section between the ends of the cable is covered by plastomer. In this embodiment the cable is firmly connected to the fabric layer while at least one end of the cable, preferably both ends of the cable, are connected for example to a sensor or an electrode and an evaluation unit or a signal generating unit. The fabric layer is advantageously part of a garment as referred to before or a bandage.

The elastomer and the cable may be arranged on the inner side of the garment facing the body as well as on the outside of the garment facing away from the body.

The invention further relates to a nozzle for the common application of elastomer and a cable on the fabric layer of a garment or a bandage. The garment or, respectively, the bandage is made in accordance with the earlier described method using the nozzle. The nozzle has a nozzle body provided with two supply openings of which a first supply opening serves for the admission of elastomer and the second supply opening for the admission of the cable. However, if expedient, the elastomer and the cable may be admitted via a common supply opening.

The elastomer and the cable can be deposited on the fabric layer via the common nozzle either at the same time or after one another.

The nozzle body has a nozzle body internal space to which the first and the second supply opening lead. In addition the nozzle body has a common exit opening for the elastomer and the cable. It is possible however that there is an exit opening for the elastomer as well as one for the cable. Within the nozzle body internal space the cable comes in contact with the elastomer and is wetted by the elastomer. When the pressurized elastomer exits the nozzle also the cable is moved out of the exit opening with a force generated by the adhesion of the elastomer to the cable. This force can be sufficient to provide for a continuous exit movement of the cable and of the nozzle.

In an advantageous embodiment on, or in, the nozzle there is a drive arrangement which drives the cable so that, depending on the positioning of the drive arrangement, the cable is either pushed into the nozzle or pulled out of the nozzle.

In a further advantageous embodiment a tubular cable guide member is integrated into the nozzle body for guiding the cable. The cable guide member extends through the interior of the nozzle body and ensures that the cable comes into contact with the elastomer only after their exit from the nozzle. This has the advantage that the cable does not adhere to the elastomer within the nozzle and the cable can move out of the nozzle uninhibited by the elastomer by relatively low drive forces. The exit opening of the cable guide member is expediently arranged within, or next to, the exit opening of the nozzle. In a preferred embodiment, the end of the tubular cable guide member extends into the exit opening of the nozzle. The cable guide member is advantageously formed integrally with the nozzle body.

Further advantages and expedient embodiments are apparent from the claims, the description of the figures and the accompanying drawings.

### BRIEF DESCRIPTION OF THE DRAWINGS

It is shown in:

FIG. 1 in a cross-sectional view a nozzle for the common application of elastomer and a cable onto a fabric layer;

FIG. 2 the nozzle according to FIG. 1 with the addition of a drive arrangement for driving the cable through the nozzle;

FIG. 3 an enlarged representation of the fabric layer with elastomer and cable;

FIG. 4 a cross-sectional view of an electrically conduction cable; and,

FIG. 5 a cross-sectional view of a nozzle for the common application of elastomer and a cable.

### DETAILED DESCRIPTION OF THE INVENTION

In the figures identical components are designated by the same reference numerals.

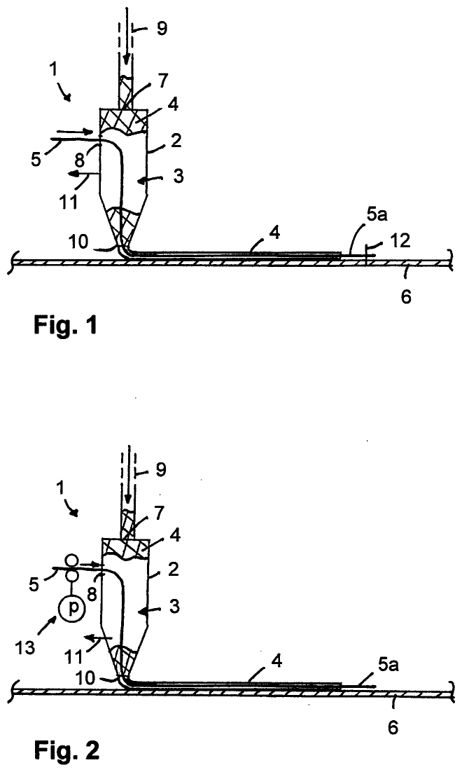

FIG. 1 shows a nozzle 1 with a nozzle body 2 with an elastomer 4 and a cable 5 disposed in the interior space 3 of the nozzle body 2 for deposition on a fabric layer 6. The fabric layer 6 is preferably arranged horizontally on work table. The nozzle is moved by means of a suitable nozzle moving arrangement across the top side of the fabric layer 6 while, at the same time, the elastomer 4 and the cable 5 are moved out of the nozzle 1 and deposited on the top side of the fabric layer 6.

The cable 5 is preferably an electrically conductive cable suitable for the transmission of electrical signals. By the elastomer 4 the cable 5 is firmly connected to the fabric layer 6. In particular, the cable 5 is surrounded by the elastomer 4 and is protected thereby from outer influences such as for example humidity. The cable 5 is advantageously elastic in the longitudinal direction by at least 1.6 times its length. Via the cable 5 for example electrical signals can be transmitted between an electrode or a sensor which is or are integrated into the fabric layer 6 and an evaluation unit or a signal generating unit. By the sensor or, respectively, the electrode for example vital parameters of a person wearing a garment or a bandage including the fabric layer 6 described earlier can be transmitted. It is also possible to transmit via the cable 5 electrical pulses to an electrode in the fabric layer 6 via which pulses individual muscle areas can be stimulated.

The garment of which the fabric layer is an integral part is for example some sport clothing such as a T-shirt or a sport pants. If it is a bandage of which the fabric layer 6 is an integral part it is in particular a sleeve-like joint bandage for example a knee bandage or an elbow bandage.

The nozzle body 2 is provided at its top with a first supply opening 7 for the addition of the elastomer 4 via a supply line 9. In the side wall of the nozzle body 2 there is a second supply opening 8 via which the cable 5 is moved into the interior 3 of the nozzle body 2. The nozzle body 2 narrows downwardly and is provided at its lower end with a common exit opening 10 for the elastomer 4 and the cable 5.

Via the supply line 9 pressurized flowable elastomer 4 is conducted into the nozzle body interior 3 into which also the cable 5 is transported via the second supply opening 8 in the side wall of the nozzle body 2. In the interior 3 of the nozzle body 2 the cable 5 is joined by the elastomer 4 and together they leave the nozzle body interior 3 via the exit opening 10 to be applied to the top side of the fabric layer 6. The travel directions of the elastomer through the supply line 9 and of the cable 5 at the side into the nozzle body interior are designated in the figure by arrows,

The nozzle 1 is moved over the fabric layer 6 in such a way that the exit opening 10 at the bottom is disposed at a small distance from the top side of the fabric layer 6. The nozzle 1 is positioned advantageously on an arrangement by which the nozzle 1 is moved horizontally in the direction of the arrow 11 over the stationary fabric layer 6. Upon leaving the exit opening 10 a worm is formed on the top side of the fabric layer 6 consisting of elastomer 4 and the cable 5, which is preferably completely surrounded by the elastomer 4. The elastomer is applied in a flowable state and can enter into the pores of the fabric layer 6. After hardening, a firm connection of the elastomer 4 including the cable with the fabric layer is established.

The end 5a of the cable 5 is at the beginning of the process, after having been pulled through the nozzle 2 and after having exited the exit opening 10, free of any elastomer 4 fixed to the fabric layer 6 or to a working table on which the fabric layer is disposed. To this end a fixing element 12 is provided, for example a clamp or similar, which engages the end 5a of the cable, as shown in FIG. 1. Wherein the nozzle 2 is moved in the direction of the arrow 11 over the fabric layer 6, the cable 5 is moved, because of its fixation by means of the fixing element 12, into the nozzle body 2 and is pulled out of the exit opening 10.

The end 5a of the cable 5 which is free of any elastomer can be connected for example to a sensor, to an electrode or to an evaluation unit or a signal generating unit.

In a corresponding way an opposite second end of the cable can also be kept free of any elastomer. To this end, the elastomer supply is shortly interrupted.

The elastomer 4 is supplied under pressure via the supply line 9 to the interior 3 of the nozzle body 2 in a heated flowable state and deposited via the exit opening 10 onto the fabric layer 6. Upon cooling the elastomer hardens but maintains a certain elasticity also in a cold state. And since also the cable 5 is elastic the fabric layer can expand and contract elastically also after the application of the elastomer 4 and the cable 5.

FIG. 2 shows an embodiment of a nozzle of essentially the same design as that shown in FIG. 1, but which is provided additionally with a drive arrangement 13 for the cable 5. The drive arrangement 13 comprises an electric motor, a driven roller and a passively co-rotating engagement roller. The driven roller is driven by the electric motor and the cable 5 is engaged between the two rollers. The drive arrangement 13 is arranged ahead of the nozzle body 2 so that the cable 5 is moved by the drive arrangement into the interior 3 of the nozzle body 2. Basically, the drive arrangement 13 may also be provided at the exit opening of the nozzle body 2 for pulling the cable 3 out of the interior 3 of the nozzle body 2. The drive arrangement 13 may be attached to the nozzle body 2 or an arrangement supporting the nozzle body 2.

The speed with which the cable 5 is driven by the drive arrangement 13 corresponds in particular to the relative speed between the nozzle 1 and the fabric layer 6. This ensures a continuous uniform exit of the cable 5 and the elastomer 4 via the exit opening 10 in the lower area of the nozzle 1. In this way the cable 5 can be applied to the fabric layer 6 without stretching or compression. It is advantageous that, in this way, fixing of the cable 5 to the fabric layer 6 or an arrangement for supporting the fabric layer can be omitted.

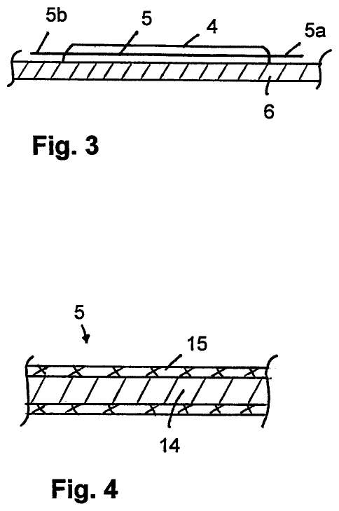

FIG. 3 shows the fabric layer 6 with a worm-like elastomer enclosing a cable 5 applied thereto in an enlarged representation. At both ends of the elastomer 4 the ends 5a and 5b of the cable 5 project from the elastomer 4. They are blank and free of any elastomer and can be used for connection to a sensor, an electrode or an evaluation unit or signal generating unit.

FIG. 4 shows a longitudinal cross-sectional view of the cable 5 which consists of a rubber core of a rubber material 14 and a surrounding metal net 15. The metal net 15 is in the form of a wire plait which is electrically conductive and suitable for the transmission of electrical signals. The inner rubber core 14 can be highly expanded and after a linear elastic expansion returns to its original length. The metal net 15 in the form of a wire plait can also be expanded in the same manner and returns to its original state when external forces are no longer present either as a result of its own elastic behavior or via the rubber core.

The linear-elastic expansion of the cable 5 is at least 60% that is 1.6 times the unexpanded original length.

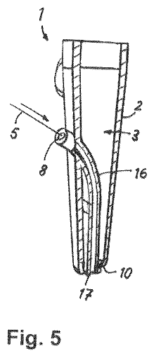

FIG. 5 shows an embodiment of a nozzle 1 wherein a tubular cable guide member 16 is integrated into the nozzle body 2 of the nozzle 1 for guiding the cable 5 through the interior 3 of the nozzle body 2. The supply opening 8 via which the cable 5 enters the cable guide member 16 is arranged at the front end of the cable guide member 16 at a side section of the nozzle body 2. The lower end of the curved cable guide member 16 extends into the exit opening 10 of the nozzle 1 so that the discharge opening 17 of the cable guide member 16 is disposed in the exit opening 10 of the nozzle 1. The elastomer 4 can leave the nozzle via the annular area between the outer wall of the cable guide member 16 and the inner wall of the exit opening 10.

The cable guide member 16 is formed integrally with the nozzle body 2.

What is claimed is:

1. A method wherein elastomer (4) with a cable (5) embedded in the elastomer (4) is applied onto a fabric layer (6) of a garment or a bandage including the following step:
the elastomer is applied to the fabric layer (6) in a deformable state via a nozzle (1),
the cable (5) is applied to the fabric layer (6) via the same nozzle (1) in phases at the same time together with the elastomer (4) in such a way that, in one phase, the elastomer (4) surrounds the cable (5) at least partially,
in another phase only the cable (5) but no elastomer (4) is applied via the nozzle (1) to the fabric layer (6),
wherein an end (5a, 5b) of the cable (5) that has already exited the nozzle (1) is fixed to the fabric layer (6),
and the fixing of the end (5a, 5b) of the cable (5) which has exited the nozzle (1) occurs before the method step in which the elastomer (4) and the cable (5) are applied at least in phases at the same time.

2. The method according to claim 1, wherein the elastomer (4) and the cable (5) exit the nozzle (1) via the same nozzle opening.

3. The method according to claim 1, wherein the other phase, in which only the cable (5) but no elastomer (4) is applied via the nozzle (1) to the fabric layer (6) is at the beginning or at the end of a method step in which the cable (5) exits the nozzle (1).

4. The method according to claim 1, wherein the cable (5) is moved by a drive arrangement (13) at a speed which corresponds to the relative speed between the nozzle (1) and the fabric layer (6).

5. The method according to claim 1, wherein the cable (5) is elastic with an elasticity with which the cable (5) is expandable by a value of at least 1.6 times.

6. The method according to claim 5, wherein the elastic cable (5) used consists of a rubber core (14) and a surrounding metal net (15).

7. The method according to claim 1, wherein the cable (5) is electrically conductive.

8. A garment or a bandage with a fabric layer produced in accordance with the method of claim 1, wherein the end (5a, 5b) of the cable (5), preferably both ends of the cable (5), are free of elastomer (4) and the cable section between the ends (5a, 5b) of the cable (5) are covered by elastomer (4).

9. A nozzle for the common application of elastomer and a cable on a fabric layer of a garment or a bandage which is produced according to the method of claim 1, wherein the nozzle body (2) of the nozzle (1) has a first supply opening (7) for the admission of elastomer (4) and a second supply opening (8) for the admission of the cable (5), wherein the first and the second supply openings (7, 8) lead to the interior (3) of the nozzle body (2) which is provided with an exit opening (10) for the elastomer (4) and the cable (5).

10. The nozzle according to claim 9, wherein a tubular guide member (16) is integrated into the nozzle body (2) of the nozzle (1) for guiding the cable (5).

11. The garment or bandage according to claim 8, wherein the cable (5) is elastic with an elasticity with which the cable (5) is expandable by a value of at least 1.6 times.

12. The garment or bandage according to claim 8, wherein the elastic cable (5) consists of a rubber core (14) and a surrounding metal net (15).

13. The garment or bandage according to claim 8, wherein the cable (5) is electrically conductive.
